(19) United States
(12) Patent Application Publication
(10) Pub. No.: US 2009/0326419 A1
(43) Pub. Date: Dec. 31, 2009

(54) Methods for a Movement and Vibration Analyzer

(76) Inventors: Hernan Alberto Gonzalez Rojas, Tarragona (ES); Erik Weber Jensen, Sant Pol De Mar (ES)

Correspondence Address: STURM & FIX LLP, 206 SIXTH AVENUE, SUITE 1213, DES MOINES, IA, 50309-4076, US

(21) Appl. No.: 12/442,784

(22) PCT Filed: Sep. 17, 2007
(86) PCT No.: PCT/DK07/50130

§ 371 Date: Mar. 25, 2009

(30) Foreign Application Priority Data
Sep. 26, 2006 (DK) PA200601249/P/HPI

(51) Int. Cl. A61B 5/103; G06N 5/02

(52) U.S. Cl. 600/587; 706/52

(57) ABSTRACT

The present patent describes a method for a Movement and Vibration Analyzer (MVA) based on Fast Fourier Transform spectral analysis, and empirical mode decomposition (EMD) for Hilbert transform of a timeseries recorded with an accelerometer attached to a human being or an object. The medical application is the detection of Parkinson's disease (PD) and other neurological motor disorders (Dystonias, Dyskinesias, Huntington's disease, Essential Tremor, Multiple System Atrophy (MSA), etc), which affects worldwide more than 5 million persons, where the highest percentage is in the ageing population. The industrial application is the study of vibration and maintenance of rotational devices (motors, turbines, and others which have an intrinsic sinusoidal likewise movement). An EMD is carried out on the acceleration signal which produces a collection of intrinsic mode functions (IMF), on which the Hilbert transform is carried out. A set of parameters extracted from the Hilbert Transformed signal gives information of the deviation of the discontinuities. (1) Number of peaks of the derivative of the Hilbert phase higher than a threshold and normalized to time length of the signal and sampling frequency. (2) Variance or standard deviation of the derivative of the Hubert phase, φ′_{H}(t). (3) Fractal dimension (D_{F}) of the curve (H_{R}(t), H_{I}(t)), Hilbert plane. From the power spectrum estimate of the acceleration signal, the parameters used are: (4) Mean frequency. (5) Frequencies of the N main components. These five parameters are combined using fuzzy logic or an ordinal multiple logistic regression to define the movement index (MI), an index from 0 to 100, where 0 indicates no deviation from the sinusoidal movement while increasing numbers indicate larger deviation from the sinusoidal movement.

Parkinson's disease (PD) and other neurological motor disorders (Dystonias, Dyskinesias, Huntington's disease, Essential Tremor, Multiple System Atrophy (MSA), etc), attacking principally the motor capacity of a person, affects worldwide more than 5 million persons, where the highest percentage is in the ageing population. The risk of developing Parkinson's disease increases with age, and afflicted individuals are usually adults over 40. In consequence, Parkinson's disease is a public problem of high relevance, a device that detects and evaluates the degree of this disease is desirable.

Parkinson's disease (PD) is a progressive degenerative disease of the central nervous system. Parkinson's disease occurs in all parts of the world.

While the primary cause of Parkinson's disease is not known, it is characterized by degeneration of dopaminergic neurons of the substantia nigra. The substantia nigra is a portion of the lower brain, or brain stem, that helps control voluntary movements. The shortage of dopanine in the brain caused by the loss of these neurons is believed to cause the observable disease symptoms.

Today the assessment is carried out by clinical signs which correct interpretation depends on the experience of the doctor doing the test, rendering this test highly subjective.

The shown method evaluates the existence and the degree of the motor capacity in an objective way, so its application to PD would help the diagnosis and the following-up of different treatments.

Introduction Rotational Devices

Maintenances of rotational devices are normally subject to fixed time intervals but it would be convenient to have a method to detect on time when the efficiency is decreasing or the machine exhibits any problem showed as a deviation from its pure periodic movement.

This method and apparatus gives an early warning that maintenance is needed.

The accelerometer is placed on the chassis and acceleration signal is acquired and analyzed on time, using the spectrum and the Hilbert Transform of the acceleration signal to evaluate the efficiency.

Apparatus Description

The apparatus consists of two units.

Unit 1. (Acquisition unit). Consists of an accelerometer, amplifier, A/D-converter with a micro processor and a Radio Frequency system (as for example Blue-tooth transmitter).

Unit 2. (Computer unit). Consists of a computer based system (such as a Personal Computer, Hand Held computer, Laptop . . . ) containing a Radio Frequency system (such as a Blue-tooth receiver).

The computer unit will receive the acceleration signal acquired by the accelerometer sent by Unit 1 and will process the data using the described algorithm based on the Hilbert Transform.

According to FIG. 1, the accelerometer (2), is attached to the hand or finger (1) of the subject for the study of Parkinson disease (PD) and on the chassis or rotor in the mechanical device. The analogue signal is converted to a digital signal via an AD-converter (3). A microprocessor or other calculation unit executes the analysis of the recorded acceleration signal (4), which is then shown on a display (5). The acceleration signal is bandpass filtered (6). An Emperical Mode Decomposition (EMD) generate a series of intrinsic mode frequencies (IMF) which are Hilbert transformed (7) and a set of N parameters (8) are extracted for the calculation of the index (11). A spectral analysis (9) is carried out as well from which M parameters are extracted (10). The index is defined as a combination of both parameter set (11).

Subject Movement Tests

The test for Parkinson's disease and Effect site evaluation are carried out the following way. The accelerometer in unit 1 is attached with a Velcro strap to the hand of the patient. The patient is asked to perform circular like movements.

The main and shared feature of all the movements is their intrinsic periodicity. These movements, some of them accepted as a clinical criteria by the doctors as representative of Parkinson disease, are:
(1) Tapping. The patient moves alternatively the hand between two fix points.
(2) Circular movement around the face (washing-face like)
(3) Finger tapping. The accelerometer is placed on the index finger while it is draws near and far from the thumb. As picking up or leaving something with both fingers.
(4) Transversal finger movement from the nose. The hand of the patient approaches and comes far from the nose alternatively.
(5) Hand trembling. The patient must try to keep the hand still while the accelerometer is positioned.

Independent of the application, unit 1 sends the acceleration of the movements trajectories to unit 2 via a radio link. Unit 2 has a built in radio receiver and a CPU to analyze the acceleration signal with the Hilbert transformation combined with the spectrum of the acceleration subsequently calculate the movement index (MI). The MI is a unitless scale ranging from 0 to 100 achieved by combining a set of sub-parameters of the Hilbert Transform and the power spectrum.

Brief Description of the Algorithm

The algorithm, applied to the acceleration signal, consists of the following steps, FIG. 1:
(1) The acceleration signal is acquired with the apparatus described before.
(2) The spectrum of the acceleration signal is calculated. Via a parametric method (as for example: Autoregressive analysis . . . ) or non parametric method (as for example: the FFT)
(3) The acceleration signal is filtered through a band pass filter. The empirical mode decomposition (EMD), producing a collection of intrinsic mode functions (IMF), to which the Hilbert Transform is applied.
(4) A set of parameters, described below, are extracted from the spectrum analysis and the Hilbert Transform.
(5) All parameters have significant information about the recorded acceleration and are combined to get the best performance. The combination methods are a Fuzzy logic inference system and different statistical methods such as ordinal regression.

Description of the Parameters

The acceleration signal is a real signal, captured with the accelerometer.

An EMD is carried out on the acceleration signal which produces a collection of IMF, on which the Hilbert transform is carried out, producing a complex signal.

As any complex signal it can be written as a Real (H_{R}(t)) and Imaginary (H_{I}(t)) parts or into a Modulus (|H(t)|) and Phase signal (φ_{H}(t)).

Henceforth φ_{H}′(t) is defined as the derivative of φ_{H}(t), being this signal one of the most important source of information about the movement performance. The radian phase signal φ_{H}(t) has been unwrapped by changing absolute jumps greater than π to their 2π complement, before applying the derivative, to make the phase continuous across 2π phase discontinuities.

The information of the acceleration extracted with the Hilbert Transform is complemented by the evaluation of the frequency contents if the acceleration signal, by means of it spectrum (calculated by parametric or non-parametric methods).

A set of N parameters extracted from the Hilbert Transformed signal gives information of the deviation of the discontinuities.
(1) Number of peaks of the derivative of the Hilbert phase higher than a threshold (normalized to time length of the signal and sampling frequency)

Number peaks φ_{H}′(t)≧threshold
(2) Variance or standard deviation of the derivative of the Hilbert phase, φ′_{H}(t).
(3) Fractal dimension (D_{F}) of the curve (H_{R}(t), H_{I}(t)), Hilbert plane.

From the power spectrum estimate of the acceleration signal, the M parameters used are:
(1) Mean frequency.
(2) Frequencies of the N main components.

### EXAMPLE OF APPLICATION

This section presents an example of the Hilbert Transform performance applied to Parkinson Disease and Rotational devices to show why this transform was selected to be used in these applications.

FIG. 2.a shows the acceleration signal from a normal subject doing one of the test movements (washing face like movement) with the accelerometer placed on the right hand. The spectrum of the acceleration signal is depicted in FIG. 2c.

The acceleration signal is filtered through a band pass filter, FIG. 2b, and then the Hilbert Transform is applied. FIG. 2d and FIG. 2e contain the curve of the Hilbert plane (H_{R}(t), H_{I}(t)) and the derivative of the phase Hilbert Transform, respectively.

FIG. 3 shows the signals and transforms for the same test, as in FIG. 2, collected from a Parkinson disease patient.

As an another example, FIG. 4, shows the effect of treatment with drugs (in this case L-Dopamine) on Parkinson disease, expressed on the derivative of Hilbert Transform's phase.

Combined Methods Description

Each of the subparameters as single parameters has prediction capacity of Parkinson's disease, correlates to the effect site concentration, and the description of the rotational device performance.

However, by combining the parameters, sensitivity and specificity are increased.

From the set of parameters detailed in section 006 several indexes are created from their combinations using one of these methods:
(1) Combining indexes using a Fuzzy Inference System. The adjustment of the Fuzzy is done by means of an ANFIS (Artificial Neural Fuzzy Inference System) algorithm.
(2) Ordinal logistic regression.
(3) Discriminate Analysis.
(4) Artificial Neural Network.

For the study of the Parkinson, different indexes will be created:
Combining the information from the power spectrum and the Hilbert Transform, relating this information with the corresponding medical criteria (physician criteria based on several tests and scores) about the existence of a neurological motor disorder.
Combining information to get the best correlation with the concentration of different treatment drugs (as L-dopamine) to control neurological motor system disorders.

An other possible application of the method is an evaluation of the effect site concentration (ES) of drugs on the subjects motoric system for people driving or manipulating machines.

The parameters will be combined based on a database adjusting with the before mentioned parameters versus different concentrations of alcohol and medical criteria about the control of the subject on their voluntary movements.
The application of the EMD has been described in the article (D1) “Empirical mode decomposition: a novel technique for the study of tremor time series by E Rocon de Lima et al, Med Bio Eng Comput (2006) 44: 569-582. This article describes how the EMD is applied to data recorded from gyroscopes attached to the arm of the patient.
The present method is significantly different from the method described in D1. First of all the method assesses the deviation from a sinusoidal movement. Secondly, the number of peaks of the derivative of the Hilbert phase higher than a threshold is a used as a main input parameter to one of the functions used to define the index of tremor. The methods used for combining the parameters are for example, but not limited to, an ANFIS or a multiple logistic regression.

Hilbert Transformation Description

The Hilbert Transform of an infinite continuous signal f(t) is defined as:

$$H \{ f ( t ) \} ≡ \frac{1}{π} {\int }_{- ∞}^{∞} f ( s ) \frac{1}{t - s} d s$$

The implementation of the Hilbert Transform of finite length digital signal can be calculated by means of the FFT (Fast Fourier Transform) as shown schematically below.

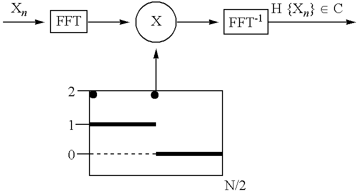

H{xn}=H_{R}{xₙ}+H_{I}{xn}=|H{xn}|·φ_{H}{xn}

H{xₙ}=FFT⁻¹(FFT(xₙ)*Wₙ) where

$$Function {H}_{n}$$

$${W}_{n} = \{ \begin{matrix}2 + j 0 ; & n = 0 , n = N / 2 \\ 1 + j 0 ; & 1 \leq n \leq N / 2 - 1 \\ 0 + j 0 ; & N / 2 + 1 \leq n \leq N - 1\end{matrix} \\ where j = \sqrt{- 1}$$

### DESCRIPTION OF FIGURES

FIG. 1. Block diagram of method and apparatus.

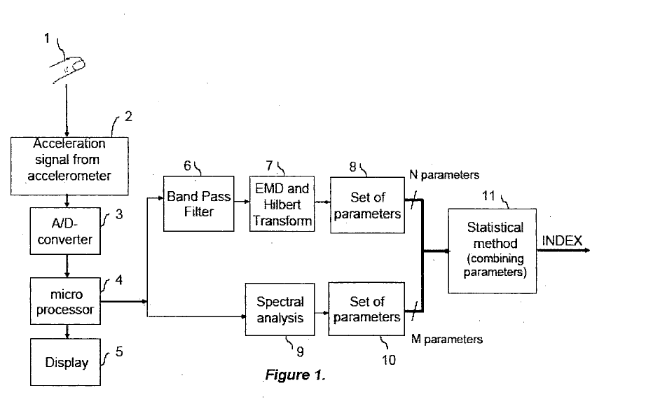

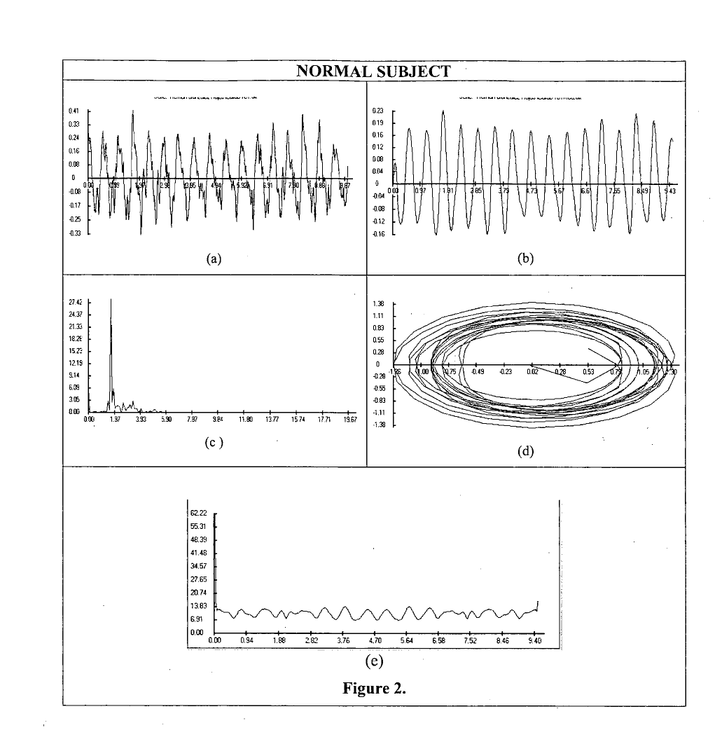

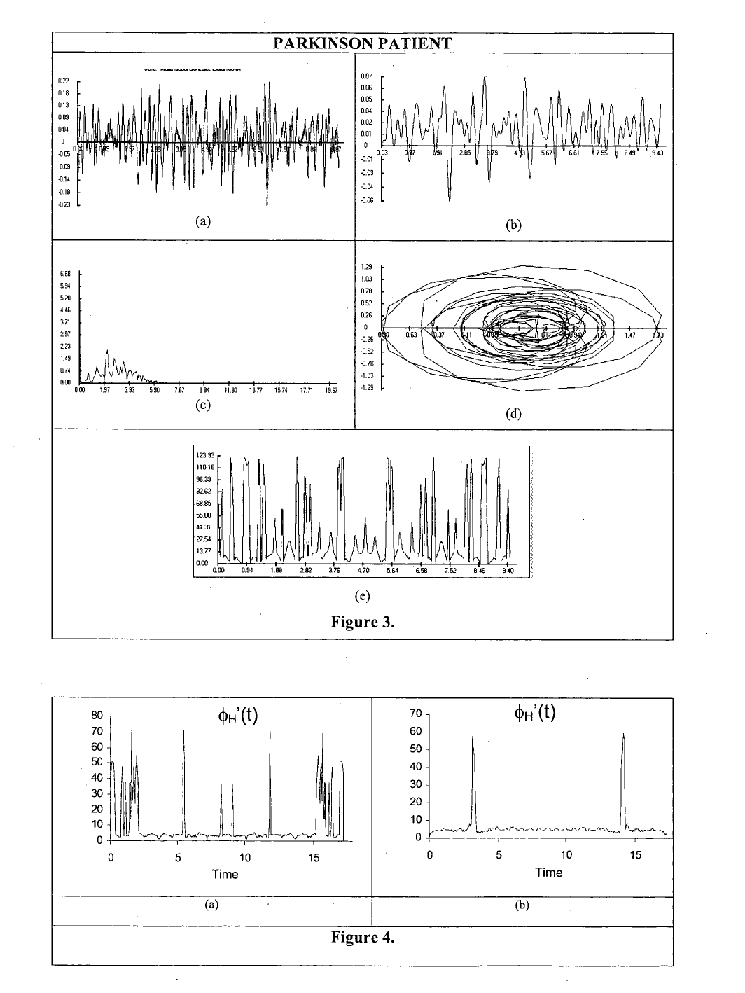

FIG. 2. Acceleration signal and Transformed signals. NORMAL SUBJECT.
(a) Acceleration signal.
(b) Filtered acceleration signal.
(c) Spectrum of acceleration signal.
(d) Hilbert Plane (H_{R}(t), H_{I}(t)) acceleration signal.
(e) Hilbert Phase derivative φ_{H}′(t) acceleration signals.

FIG. 3. Acceleration signal and Transformed signals. PARKINSON PATIENT.
(a) Acceleration signal.
(b) Filtered acceleration signal.
(c) Spectrum acceleration signal.
(d) Hilbert Plane (H_{R}(t), H_{I}(t)) acceleration signal.
(e) Hilbert Phase derivative φ_{H}′(t) of acceleration signals.

FIG. 4. φ_{H}′(t) of acceleration signal of a Parkinson patient making a tapping movement.
(a) before administering L-Dopamine.
(b) 20 min after administering L-Dopamine.

1. A Method for determining the deviation from periodic or sinusoidal like motion, termed the Movement and Vibration Analyzer (MVA) based on extraction of parameters from a digitally sampled time series called acceleration signal and registered by an accelerometer attached to the individual or the object to be analyzed where the method comprises:
(a) calculation of the power spectrum of the acceleration signal;
(b) processing of the acceleration signal with empirical mode decomposition generating a collection of intrinsic mode functions, to which the Hilbert transformation is applied;
(c) calculation of the number of peaks of the derivative of the Hilbert phase higher than a threshold value;
(d) determining the variance or standard deviation of the derivative of the Hilbert phase;
(e) determining the fractal dimension of the curve in the Hilbert plane;
(f) determining the mean frequency of the power spectrum;
(g) determining the frequencies of the N main components in the power spectrum;
(h) determining the combination of the extracted parameters from the power spectrum and the Hilbert transformation by a fuzzy logic or multiple regression function that defines a scale where increasing values indicate greater deviation from the sinusoidal movement.

2. The method of claim 1, step b, further comprising using the Hilbert transformation from which the derivative of the Hilbert phase is obtained included in the algorithm.

3. The method of claim 2, step c, further comprising using the Hilbert transformation where one parameter is the number of peaks of the derivative of the Hilbert phase, which are higher than a threshold normalized to time length of the signal and sampling frequency.

4. The method of claim 1, step d, wherein the method uses the Hilbert transformation characterized by one parameter which is the variance or standard deviation of the derivative of the Hilbert phase.

5. The method of claim 1, step e, wherein the method uses the Hilbert transformation where one parameter is Fractal dimension (D_{F}) of the curve that connects the points in the Hilbert plane and where the x-axis is the real part whereas the y-axis is the imaginary part of the Hilbert transformation.

6. The method of claim 1, step f, wherein the method uses the power spectrum estimate of the acceleration characterized by the parameters mean frequency and frequencies of the N main components are derived.

7. The method of claim 3 used as input to a fuzzy logic combiner characterized by an Adaptive Neuro Fuzzy Inference System (ANFIS) where the weight of the rules were assessed by training on known values of input-output pairs; the relationship between the input parameters could also be assessed by an ordinal logistic regression (ORL); the output of the classification technique, fuzzy or ORL, concludes whether a motion disorder exists.

8. (canceled)

9. The method of claim 4, used as input to a fuzzy logic combiner characterized by an Adaptive Neuro Fuzzy Inference System (ANFIS) where the weight of the rules were assessed by training on known values of input-output pairs; the relationship between the input parameters could also be assessed by an ordinal logistic regression (ORL); the output of the classification technique, fuzzy or ORL, concludes whether a motion disorder exists.

10. The method of claim 5, used as input to a fuzzy logic combiner characterized by an Adaptive Neuro Fuzzy Inference System (ANFIS) where the weight of the rules were assessed by training on known values of input-output pairs; the relationship between the input parameters could also be assessed by an ordinal logistic regression (ORL); the output of the classification technique, fuzzy or ORL, concludes whether a motion disorder exists.

11. The method of claim 6, used as input to a fuzzy logic combiner characterized by an Adaptive Neuro Fuzzy Inference System (ANFIS) where the weight of the rules were assessed by training on known values of input-output pairs; the relationship between the input parameters could also be assessed by an ordinal logistic regression (ORL); the output of the classification technique, fuzzy or ORL, concludes whether a motion disorder exists.

12. The method of claim 7 wherein the output of the classification technique is characterized by a zero to hundred scale, where 0 indicates no deviation from normal sinusoidal motion or movement while values above 50 indicate a high probability of movement disorders.

13. The method of claim 9 wherein the output of the classification technique is characterized by a zero to hundred scale, where 0 indicates no deviation from normal sinusoidal motion or movement while values above 50 indicate a high probability of movement disorders.

14. The method of claim 10 wherein the output of the classification technique is characterized by a zero to hundred scale, where 0 indicates no deviation from normal sinusoidal motion or movement while values above 50 indicate a high probability of movement disorders.

15. The method of claim 11 wherein the output of the classification technique is characterized by a zero to hundred scale, where 0 indicates no deviation from normal sinusoidal motion or movement while values above 50 indicate a high probability of movement disorders.

16. The method of claim 12 wherein the output of the classification technique is characterized by a zero to hundred scale, where 0 indicates no deviation from normal sinusoidal motion or movement while values above 50 indicate a high probability of movement disorders.
